SEQUENCE LISTING (26 sequences)

SEQ ID NO: 1 - protein, 14 aa, Unknown
  Description of Unknown Tumor necrosis factor receptor super family peptide
KSRTGDEIILPRGL

SEQ ID NO: 2 - protein, 15 aa, Artificial Sequence
  Description of Artificial Sequence Synthetic peptide
KSRTGDEIILPRGLC

SEQ ID NO: 3 - protein, 462 aa, Artificial Sequence
  Description of Artificial Sequence Synthetic polypeptide
MNFGFSLIFLVLVLKGVQCEVKLVESGGGLVKPGGSLKLSCAASGITFSTYAMSWVRQTPEKRLEWVASITTGGNVYFPDSVKGRFTISRDNARNILYLQMSSLRSEDTAMYYCAGGITATRGTFAYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 4 - DNA, 1389 bp, Artificial Sequence
  Description of Artificial Sequence Synthetic polynucleotide
atgaacttcgggttcagcttgattttccttgtccttgttttaaaaggtgtccagtgtgaagtgaagctggtggagtctgggggaggcttagtgaagcctggagggtccctgaaactctcctgtgcagcctctggaatcactttcagtacatatgccatgtcttgggttcgccagaccccagagaagaggctggagtgggtcgcatccattactactggtggtaatgtctactttccagacagtgtgaagggccgattcaccatctccagagataatgccaggaacatcctgtacctgcaaatgagcagtctgaggtctgaggacacggccatgtattactgtgcaggaggaattacggctacaagggggacctttgcctactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 5 - protein, 238 aa, Artificial Sequence
  Description of Artificial Sequence Synthetic polypeptide
MKLPVRLLVLMFWIPASSSDVLMTQTPLSLPVSLGDQASISCRSSQSIGHSNGITYLEWYLQKPGQSPKLLIYKVSNRFSGVPDRFSGSGSGTDFTLKISRVEAEDLGVYYCFEGSHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 6 - DNA, 717 bp, Artificial Sequence
  Description of Artificial Sequence Synthetic polynucleotide
atgaagttgcctgttaggctgttggtgctgatgttctggattcctgcttccagcagtgatgtcttgatgacccaaactccactctccctgcctgtcagtcttggagatcaagcctccatctcttgcagatctagtcagagcattggacatagtaatggaatcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttgaaggttcacatgttccgctcacgttcggtgctgggaccaagctggaactgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 7 - protein, 119 aa, Artificial Sequence
  Description of Artificial Sequence Synthetic polypeptide
EVKLVESGGGLVKPGGSLKLSCAASGITFSTYAMSWVRQTPEKRLEWVASITTGGNVYFPDSVKGRFTISRDNARNILYLQMSSLRSEDTAMYYCAGGITATRGTFAYWGQGTTLTVSS

SEQ ID NO: 8 - DNA, 357 bp, Artificial Sequence
  Description of Artificial Sequence Synthetic polynucleotide
gaagtgaagctggtggagtctgggggaggcttagtgaagcctggagggtccctgaaactctcctgtgcagcctctggaatcactttcagtacatatgccatgtcttgggttcgccagaccccagagaagaggctggagtgggtcgcatccattactactggtggtaatgtctactttccagacagtgtgaagggccgattcaccatctccagagataatgccaggaacatcctgtacctgcaaatgagcagtctgaggtctgaggacacggccatgtattactgtgcaggaggaattacggctacaagggggacctttgcctactggggccaaggcaccactctcacagtctcctca

SEQ ID NO: 9 - protein, 112 aa, Artificial Sequence
  Description of Artificial Sequence Synthetic polypeptide
DVLMTQTPLSLPVSLGDQASISCRSSQSIGHSNGITYLEWYLQKPGQSPKLLIYKVSNRFSGVPDRFSGSGSGTDFTLKISRVEAEDLGVYYCFEGSHVPLTFGAGTKLELK

SEQ ID NO: 10 - DNA, 336 bp, Artificial Sequence
  Description of Artificial Sequence Synthetic polynucleotide
gatgtcttgatgacccaaactccactctccctgcctgtcagtcttggagatcaagcctccatctcttgcagatctagtcagagcattggacatagtaatggaatcacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttgaaggttcacatgttccgctcacgttcggtgctgggaccaagctggaactgaaa

SEQ ID NO: 11 - protein, 8 aa, Artificial Sequence
  Description of Artificial Sequence Synthetic peptide
GITFSTYA

SEQ ID NO: 12 - protein, 8 aa, Artificial Sequence
  Description of Artificial Sequence Synthetic peptide
ITTGGNVY

SEQ ID NO: 13 - protein, 12 aa, Artificial Sequence
  Description of Artificial Sequence Synthetic peptide
GGITATRGTFAY

SEQ ID NO: 14 - DNA, 24 bp, Artificial Sequence
  Description of Artificial Sequence Synthetic oligonucleotide
ggaatcactttcagtacatatgcc

SEQ ID NO: 15 - DNA, 24 bp, Artificial Sequence
  Description of Artificial Sequence Synthetic oligonucleotide
attactactggtggtaatgtctac

SEQ ID NO: 16 - DNA, 36 bp, Artificial Sequence
  Description of Artificial Sequence Synthetic oligonucleotide
ggaggaattacggctacaagggggacctttgcctac

SEQ ID NO: 17 - protein, 11 aa, Artificial Sequence
  Description of Artificial Sequence Synthetic peptide
QSIGHSNGITY

SEQ ID NO: 18 - protein, 3 aa, Artificial Sequence
  Description of Artificial Sequence Synthetic peptide
KVS

SEQ ID NO: 19 - protein, 9 aa, Artificial Sequence
  Description of Artificial Sequence Synthetic peptide
FEGSHVPLT

SEQ ID NO: 20 - DNA, 33 bp, Artificial Sequence
  Description of Artificial Sequence Synthetic oligonucleotide
cagagcattggacatagtaatggaatcacctat

SEQ ID NO: 21 - DNA, 9 bp, Artificial Sequence
  Description of Artificial Sequence Synthetic oligonucleotide
aaagtttcc

SEQ ID NO: 22 - DNA, 27 bp, Artificial Sequence
  Description of Artificial Sequence Synthetic oligonucleotide
tttgaaggttcacatgttccgctcacg

SEQ ID NO: 23 - protein, 324 aa, Artificial Sequence
  Description of Artificial Sequence Synthetic polypeptide
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 24 - protein, 107 aa, Artificial Sequence
  Description of Artificial Sequence Synthetic polypeptide
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 975 bp, Artificial Sequence
  Description of Artificial Sequence Synthetic polynucleotide
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 26 - DNA, 324 bp, Artificial Sequence
  Description of Artificial Sequence Synthetic polynucleotide
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

(19) United States
(12) Patent Application Publication
(10) Pub. No.: US 2012/0027763 A1
(43) Pub. Date: Feb. 2, 2012

(54) Antibody for Targeted induction of Apoptosis, CDC and ADCC mediated killing of Cancer cells, TBL-CLN1

(75) Inventors: Koteswara Rao KOLLIPARA, Hyderabad (IN); Ramesh Babu BATCHU, Royal Oak, MI (US)

(73) Assignee: TRANSGENE BIOTEK LTD., Medak District (IN)

(21) Appl. No.: 13/041,203

(22) Filed: Mar. 4, 2011

(30) Foreign Application Priority Data
Mar. 4, 2010 (IN) 568/CHE/2010

(51) Int. Cl. C07K 16/30; A61K 39/395; A61P 35/04; G01N 33/566

(52) U.S. Cl. 424/138.1; 436/501; 530/326; 530/387.7; 530/387.3

(57) ABSTRACT

Antibody for targeted induction of Apoptosis, CDC and ADCC mediated killing of Cancer cells, TBL-CLN1, is disclosed. The antibodies, TBL-CLN1, are monoclonal antibodies which can specifically target and bind to the epitope of SEQ ID NO:1 expressed on cancer cells which further leads to killing of cancer cells. TBL-CLN1 is not conjugated to toxin or cytotoxic molecules, and provides selective killing of cancer cells just by binding to cancer cell surface. Also, disclosed herein is SEQ ID NO: 2 which is an engineered epitope which comprises of polypeptide sequence of SEQ ID NO: 1 and a cysteine residue which is added at the carboxyl end of the SEQ ID NO: 1. The epitope of SEQ ID NO: 2 is used to generate monoclonal antibodies described herein.

### CROSS-REFERENCE TO PRIOR APPLICATION

This application claims priority to Indian Application No.: 568/CHE/2010 filed Mar. 4, 2010.

### FIELD OF INVENTION

The present invention relates to the field of immunotherapy for cancers, and more particularly to monoclonal antibodies which specifically target an epitope expressed on cancer cells.

### BACKGROUND OF INVENTION

Important breakthroughs in cancer therapy include clinical application of antibodies. The therapeutic strategy relies on the deliberate and selective induction of apoptosis or killing by antibody-dependent cell-mediated cytotoxicity (hereinafter referred to as ADCC) and complement-dependent cytotoxicity (hereinafter referred to as CDC) of malignant cells. Importantly, therapy-resistance in cancer is frequently associated with de-regulation in the mechanisms that control apoptosis. However, cancer cells are often reliant on these molecular aberrations for survival. Therefore, selective induction of apoptosis, CDC and ADCC in cancer cells but not normal cells is a challenge to be addressed.

Induction of apoptosis in tumor cells by tumor necrosis factor [TNF]-related apoptosis-inducing ligand (hereinafter referred to as TRAIL) is a promising therapeutic principle in oncology. Programmed cell death, known as apoptosis, is an essential cellular homeostasis mechanism that ensures correct development and function of multi-cellular organisms. The pivotal importance of correct execution of apoptosis is apparent from the many human diseases with aberrancies in apoptosis, including cancer.

One possible treatment for cancer involves monoclonal antibodies (mAb) that bind only to cancer cell-specific antigens and induce an immunological response against the target cancer cell. Such mAb could also be modified for delivery of a toxin, radioisotope, cytokine or other active conjugate. It is also possible to design bispecific antibodies that can bind with their fragment antigen binding (hereinafter referred to as Fab) regions both to target antigen and to a conjugate or effector cell.

Monoclonal antibody drugs are a relatively new innovation in cancer treatment. While several monoclonal antibody drugs are available for treating certain cancers, the best way to use these new drugs isn't always clear. The immune system attacks foreign invaders in our body, but it doesn't always recognize cancer cells as enemies. A monoclonal antibody can be directed to attach to certain parts of a cancer cell. In this way, the antibody marks the cancer cell and makes it easier for the immune system to find.

Monoclonal antibody developed to the specific cancer cell surface target can kill the cell with or without toxin attached just by binding to cell surface target. The antibody can initiate lysis of the cancer cell through apoptosis, CDC and ADCC. Monoclonal antibody therapy can be used to destroy malignant tumor cells and prevent tumor growth by blocking specific cell receptors or by delivering a conjugated toxin.

There is a need for an antibody which can selectively target and induce killing of cancer cells.

### OBJECT OF INVENTION

The principal object of this invention is to provide a method for targeted and selective killing of cancer cells, wherein the killing may be induced by apoptosis, ADCC and CDC of cancer cells.

Another object of the invention is to provide a method for selective elimination of cancer cells without affecting the normal cell population.

### STATEMENT OF INVENTION

Accordingly the invention provides a polypeptide sequence of SEQ ID NO: 2, which is an engineered polypeptide sequence comprising of SEQ ID NO: 1 and a cysteine residue at the carboxyl end of SEQ ID NO: 1, and SEQ ID NO: 1 is an epitope on the Tumor necrosis factor receptor super family (hereinafter referred to as TNFRSF17).

There is also provided a polynucleotide sequence which is characterized in that to encode the polypeptide sequence of SEQ ID NO: 2.

In another embodiment, the invention provides an antibody that specifically binds to a polypeptide sequence of SEQ ID NO: 1 and/or SEQ ID NO: 2, wherein SEQ ID NO: 1 is an epitope on the TNFRSF17 and SEQ ID NO: 2, is an engineered polypeptide produced by adding a cysteine residue at the carboxyl end of SEQ ID NO: 1.

In another embodiment, the invention provides a polynucleotide sequence that encodes an antibody which binds to a polypeptide sequence selected from the group consisting of SEQ ID NO: 1 and SEQ ID NO:2, wherein the polynucleotide sequence comprises of the sequence of SEQ ID NO: 4 and SEQ ID NO: 6.

In another embodiment, the invention provides an antibody comprising a heavy chain polypeptide sequence of SEQ ID NO:3 and a light chain polypeptide sequence of SEQ ID NO:5, wherein the variable heavy chain polypeptide sequence is of SED ID NO:7, variable light chain polypeptide sequence is of SED ID NO:9, variable heavy chain CDR1 (complementarity determining region 1) is of SEQ ID NO:11, variable heavy chain CDR2 (complementarity determining region 2) is of SEQ ID NO:12, variable heavy chain CDR3 (complementarity determining region 3) is of SEQ ID NO:13, variable light chain CDR1 is of SEQ ID NO:17, variable light chain CDR2 is of SEQ ID NO:18, variable light chain CDR3 is of SEQ ID NO:19.

In another embodiment, the invention provides an antibody, specifically a monoclonal antibody, which can induce complement dependent cytotoxicity (CDC) of cancer cells expressing TNFRSF17, apoptosis of cancer cells expressing TNFRSF17 and/or antibody dependent cellular cytotoxicity (ADCC) of cancer cells expressing TNFRSF17.

In another embodiment, the invention provides a pharmaceutical composition comprising the antibody, specifically monoclonal antibody, and a pharmaceutically acceptable carrier.

In yet another embodiment, the invention provides a method of eliciting an immune response, in a mammalian subject, directed against the polypeptide sequence of SEQ ID NO: 1 or SEQ ID NO: 2, wherein the method comprises of administering the polypeptide sequence of SEQ ID NO: 1 or SEQ ID NO: 2 into the mammalian subject.

In another embodiment, the invention provides a method of treating cancer by administering a therapeutically effective amount of the antibody, specifically monoclonal antibody, of the present invention, wherein the cancer is Multiple myeloma (hereinafter referred to as MM), Colon cancer (hereinafter referred to as CC) or Non-Hodgkin's lymphoma (hereinafter referred to as NHL).

In another embodiment, the invention provides a diagnostic method for diagnosing the presence of TNFRSF17 or cell expressing TNFRSF17 in a sample by contacting the sample with antibody of the present invention, under conditions that allow for formation of a complex between said antibody and TNFRSF17, and detecting the formation of the complex in the sample, wherein the sample is any mammalian tissue sample or an extract of any mammalian tissue sample suspected of having cancer, wherein the cells expressing TNFRSF17 include MM cancer cells, CC cancer cells and NHL cancer cells.

In yet another embodiment, the invention provides a diagnostic kit for detecting the presence of TNFRSF17 or cell expressing TNFRSF17 in a sample, wherein said diagnostic kit comprises of antibody, specifically monoclonal antibody, of the present invention.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

This invention is illustrated in the accompanying drawings, throughout which like reference letters indicate corresponding parts in the various FIGURES. The embodiments herein will be better understood from the following description with reference to the drawings, in which:

FIG. 1 is a dose response graph depicting the results of direct cytotoxicity and dose dependent assay in IM9 cells with 1.2 mg/mL of TBL-CLN1 sample;

FIG. 2 is a dose response graph depicting the results of direct cytotoxicity and dose dependent assay in IM9 cells with 2.4 mg/mL of TBL-CLN1 sample;

FIG. 3 is a dose response graph depicting the results of direct cytotoxicity and dose dependent assay in RPMI-8266 cells with 1.2 mg/mL of TBL-CLN1 sample;

FIG. 4 is a dose response graph depicting the results of direct cytotoxicity and dose dependent assay in RPMI-8266 cells with 2.4 mg/mL of TBL-CLN1 sample;

FIG. 5 is a dose response graph depicting the results of direct cytotoxicity and dose dependent assay in SW620 cell line with 1.2 mg/mL of TBL-CLN1 sample;

FIG. 6 is a dose response graph depicting the results of direct cytotoxicity and dose dependent assay in SW620 cell line with 2.4 mg/mL of TBL-CLN1 sample;

FIG. 7 is a dose response graph depicting the results of direct cytotoxicity and dose dependent assay in LOVO cell line with 2.4 mg/mL of TBL-CLN1 sample;

FIG. 8 is a dose response graph depicting the results of complement dependent cytotoxicity in IM9 cells;

FIG. 9 is a dose response graph depicting the results of complement dependent cytotoxicity in RPMI-8266 cells;

FIG. 10 is a dose response graph depicting the results of complement dependent cytotoxicity in SW620 cell line; and

FIG. 11 is a dose response graph depicting the results of complement dependent cytotoxicity in LOVO cell line.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

Embodiments of the present invention are directed towards an antibody and a method for targeting and killing of cancer cells including; Colon cancer, Multiple Myeloma and Non-Hodgkin's lymphoma cancer cells. The main focus of the present invention is to selectively target and kill cancer cells by monoclonal antibodies produced against a unique surface antigen over expressed on Colon cancer (CC), Multiple Myeloma (MM) and Non-Hodgkin's lymphoma (NHL) cancer cells. According to a non limiting aspect, the invention focuses on enhancing the immunogenicity of the surface antigen by adding a cysteine residue to the carboxyl end of the 14 amino acid long extra-cellular antigenic peptide epitope.

Tumor necrosis factor receptors (TNFR) are single transmembrane-spanning glycoproteins that bind cytokines and trigger multiple signal transduction pathways. Tumor necrosis factor (TNF) is a pro-inflammatory cytokine whose role is established in the pathogenesis of malignant diseases like cancer. TNF has been found to have a pro-cancerous effect and gene polymorphisms which increase or decrease TNF production results either in increased risk or protective effect on a number of different cancers and precancerous diseases including gastric cancer, lymphoma and cervical cancer.

Certain TNFR member proteins are expressed on specific cancer cells. TNFR has been detected in a number of different solid tumor types as well as hematological malignancies.

TNF is a cytokine that is produced early in the inflammatory cascade and has been shown to promote carcinogenesis. TNF has a wide range of activities in cancer. TNF has converse actions that induce a number of pro-inflammatory genes, which the tumors utilize to promote cancer such as cytokines, angiogenic factors and MMPs. These factors contribute to tumor formation, growth, invasion and metastasis to other sites. Many of the actions of TNF may occur by the stimulation of stromal tissue, tumor-associated macrophages and fibroblasts. These cells may then produce inflammatory cytokines including TNF itself, as well as some of the angiogenic factors described above, contributing to tumor proliferation and invasion.

Anti-TNF mAbs are widely used against a range of cancers like breast cancer, metastatic colorectal cancer, NHL and other lymphoblastic malignancies. However, the anti-TNF monoclonal antibodies are chimeric antibodies either conjugated to a toxin moiety or a radioactive substance which kill targeted cancer cells by selectively binding to TNFRs and rendering the toxic or radioactive effect on the tumor cells with specific and non-specific surface antigen epitope, thus affecting the bystander lymphocytes.

The embodiments disclosed herein aims at targeting a certain class of surface antigens expressed on Colon cancer, Multiple Myeloma and Non-Hodgkin's lymphoma cancer cells. The surface antigen is TNFRSF17. The invention ensures specific targeting of lymphocytes which express TNFRSF17. The surface antigen epitope on TNFRSF17 is a 14 amino acid sequence of the order as provided under SEQ ID NO:1, from amino terminal to carboxyl terminal end.

In an embodiment, the addition of a cysteine residue to the carboxyl end enhances the immunogenicity of the antigen epitope. The addition of cysteine results in an antigenic peptide of 15 amino acid sequence (also referred to as TNFRS17-epag) of the order as provided under SEQ ID NO:2, from amino terminal to carboxyl terminal end. The antigen epitope of SEQ ID NO:1 is modified and purified by techniques known in the art.

The disclosed embodiments underline the production of monoclonal antibodies, generated against cancer cells including Colon cancer, Multiple Myeloma and Non-Hodgkin's lymphoma cancer cells from patients and cell lines but not normal tissues according to a non limiting aspect of the present invention. The monoclonal antibody has a binding domain specific for TNFRS17-epag. The therapeutic antibodies specifically bind to the TNFRS17 on the targeted cancer cells and kill them mediated by targeted induction of apoptosis, antibody-dependent cytotoxic immune responses and complement-dependent cytotoxic immune response. This antigen specific antibody response does not harm the bystander B cells and other immune cells like T cell, NK cells, mast cells and stimulated neutrophills.

In an embodiment, an epitope of the polypeptide sequence of SEQ ID NO: 2 is disclosed. SEQ ID NO: 2 is an engineered epitope which comprises of polypeptide sequence of SEQ ID NO: 1 and a cysteine residue which is added at the carboxyl end of the SEQ ID NO: 1. The polypeptide sequence of SEQ ID NO: 2 is more immunogenic due to the addition of cysteine residue. The epitope of SEQ ID NO: 2 is further used to generate antibodies described herein.

### Identification of the Epitope of SEQ Id NO: 1 and Preparation of Engineered Epitope of SEQ ID NO: 2:

The 15 amino acid sequence of SEQ ID NO: 2, TNFRSF17-epag, of the present invention can be produced by adding a cysteine residue to carboxyl end of the 14 amino acid sequence of SEQ ID NO: 1. SEQ ID NO: 2 is more immunogenic and may be used to produce antibodies, specifically monoclonal antibodies. The monoclonal antibodies of the present invention can specifically bind to polypeptide sequence of SEQ ID NO: 2.

The polypeptide sequence of SEQ ID NO: 2 may be prepared by techniques known in the art including chemical peptide synthesis and genetic engineering.

SEQ ID NO: 1 is the 14 amino acid sequence which is an epitope present on the extracellular domain of TNFRSF17. TNFRSF17 is a unique cell surface antigen expressed on MM, CC and NHL cancer cells. The monoclonal antibodies of the present invention can specifically bind to polypeptide sequence of SEQ ID NO: 1 present on MM, CC and NHL cancer cells and induce killing of such cancer cells.

### Production of the Antibodies:

The antibody disclosed herein can be produced by various methods familiar to those skilled in the art, such as hybridoma methodology.

In an embodiment of the invention, the antibodies are produced by the conventional hybridoma technology, wherein a mouse is immunized with an antigen. The spleen cells are then isolated and fused with myeloma cells lacking Hypoxanthine-guanine phosphoribosyltransferase (HGPRT) expression. The hybrid cells are selected using hypoxanthine, aminopterin and thymine (HAT) containing media.

The antibodies disclosed herein can also be made by other methods familiar to those skilled in the art including recombinant DNA methods.

The disclosed monoclonal antibodies against the polypeptide sequence of SEQ ID NO: 2 (TNFRS17-epag), which can induce targeted killing of cancer cells are referred to as TBL-CLN1.

### Polynucleotide and Amino Acid Sequences of TBL-CLN1

The disclosed antibodies, in an embodiment, comprises of at least two heavy (H) chains and two light (L) chains inter-connected by disulfide bonds, or an antigen binding fragment. Each heavy chain sequence comprises of a leading sequence, heavy chain variable sequence (abbreviated as VH) and a heavy chain constant sequence. Each light chain comprises of a leading sequence, light chain variable sequence (abbreviated herein as VL) and a light chain constant sequence.

The VH and VL regions can be further subdivided into regions of hypervariability, termed as complementarity determining regions (CDR) which are interspersed with regions that are more conserved, termed as framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies mediate the binding of the antibodies to host tissues or factors, including various cells of the immune system (e.g. effector cells) and components of the complement system.

The cDNAs encoding the variable region and constant region, including heavy and light chains, of the antibodies were amplified by PCR, cloned, and sequenced. The polypeptide and polynucleotide sequences of the heavy and light chains of TBL-CLN1 are as follows:

SEQ ID NO:3 and SEQ ID NO:5 are the amino acid sequences for the heavy chain and light chain of TBL-CLN1, respectively.

SEQ ID NO:4 and SEQ ID NO:6 are the nucleotide sequence encoding the heavy chain and light chain of TBL-CLNI, respectively. SEQ ID NO:7 is the amino acid sequences of variable region of heavy chain of TBL-CLN1. SEQ ID NO:9 is the amino acid sequences of variable region of light chain of TBL-CLN1. SEQ ID NO:8 is the nucleotide sequence encoding the variable region of heavy chain of TBL-CLN1. SEQ ID NO:10 is the nucleotide sequence encoding the variable region of light chain of TBL-CLN1. The variable heavy chain (complementarity determining region) CDR regions of TBL-CLN1 comprises of CDR1, CDR2 and CDR3 which has the amino acid sequence of SEQ ID NO:11, SEQ ID NO:12 and SEQ ID NO:13. The nucleotide sequence encoding SEQ ID NO:11, SEQ ID NO:12 and SEQ ID NO:13 are SEQ ID NO: 14, SEQ ID NO: 15 and SEQ ID NO: 16. The variable light chain (complementarity determining region) CDR regions of TBL-CLN1 also comprises of CDR1, CDR2 and CDR3 which has the amino acid sequence of SEQ ID NO:17, SEQ ID NO:18 and SEQ ID NO:19. The nucleotide sequence encoding SEQ ID NO:17, SEQ ID NO:18 and SEQ ID NO:19 are SEQ ID NO: 20, SEQ ID NO: 21 and SEQ ID NO: 22. The constant heavy and light chain region of TBL-CLN1 has the amino acid sequence of SEQ ID NO:23 and SEQ ID NO:24, respectively. The nucleotide sequence encoding the constant heavy and light chain region of TBL-CLN1 are SEQ ID NO:25 and SEQ ID NO:26.

In an embodiment, the polynucleotide sequences SEQ ID NO:8 and SEQ ID NO:10 may be present in a vector.

In another embodiment, the vector consisting of polynucleotide sequences SEQ ID NO:8 and SEQ ID NO:10 may be present in a cell line.

### Characteristics of the Antibodies:

The antibodies of the present invention are capable of inducing targeted killing of cancer cells that express TNFRSF17 by CDC, ADCC and apoptosis.

The term “antibody”, as used herein, includes antigen-binding fragment. The term “antigen-binding fragment”, as used herein, refers to one or more fragments of an antibody that retain the ability to bind to an antigen. It's known that the antigen-binding function of an antibody can be performed by fragments of an intact antibody. The term “antigen-binding fragment” includes (ι) a Fab fragment and (u) a F(ab′)2 fragment.

In an embodiment disclosed herein, the antibodies are preferably IgG1 and may include IgG3 and IgG4 antibodies. Further, other antibody isotypes are also encompassed by the invention, including IgG2, IgM, IgA1, IgA2, secretory IgA, IgD, and IgE.

In cancer cells expressing TNFRSF17, the antibodies of the present invention may induce complement dependent cytotoxicity (CDC) in the presence of complement system, apoptosis and antibody dependent cellular cytotoxicity (ADCC) in the presence of effector cells. The antibodies may also be capable of prolonging the survival of a subject having tumor cells expressing TNFRSF17 and further be capable of depleting the cancer cells expressing TNFRSF17.

The effector cells include all the effector cells of the immune system such as NK cells, T cells, monocytes, macrophages, etc.

### Nucleic Acid

In an embodiment, a polynucleotide sequence which encodes the polypeptide sequence of SEQ ID NO: 2 is provided.

### Pharmaceutical Compositions of the Antibodies

A disclosed embodiment provides a pharmaceutical composition. The pharmaceutical composition may comprise antibody of the present invention (specifically monoclonal antibody, TBL-CLN1) and may also contain a pharmaceutically acceptable carrier.

The pharmaceutically acceptable carriers in the pharmaceutical composition include generally used carriers well known in the art including water, salt solutions, gelatins, oils, alcohols, and other excipients and auxiliaries that facilitate processing of the active compounds into preparations that may be used pharmaceutically.

The pharmaceutical composition may further consist of certain stabilizers, salts such as sodium chloride, sodium citrate and polysorbate, and water for injection. The pH of the composition may preferably range from 6 to 7.5.

The pharmaceutical composition may be administered to a patient as injections in a range of doses starting from 0.05 to 10 mg/Kg of body weight.

In addition to the antibody of the present invention, the pharmaceutical composition may also comprise of pharmaceutically acceptable diluents, preservatives, solubilizers, emulsifiers, adjuvant and/or other carriers well known in the art.

### Treatment

Further, the embodiments disclosed herein provide the use of the isolated antibody (specifically monoclonal antibody, TBL-CLN1) in the treatment of cancer.

The disclosed antibody (specifically monoclonal antibody, TBL-CLN1), in an embodiment, is used to in treating cancer by administering a therapeutically effective amount of TBL-CLN1.

The antibody (specifically monoclonal antibody, TBL-CLN1), in an embodiment, can be isolated by techniques well known in the art. The types of cancer that can be treated include but are not limited to Multiple Myeloma (MM), Colon Cancer (CC) and Non-Hodgkin's lymphoma (NHL).

The term “therapeutically effective amount” includes an amount of the composition having therapeutic effect on a subject upon administration of the composition to the subject. The composition may be administered in doses preferably ranging from 0.05 to 10 mg/Kg of body weight

Further, a method of eliciting an immune response in a mammalian subject directed against polypeptide sequence of SEQ ID NO:1 or SEQ ID NO:2, wherein the subject is administered with an immunologically sufficient amount of polypeptide sequence of SEQ ID NO: 1 or SEQ ID NO: 2, is also provided in an embodiment.

The term “immunologically sufficient amount” includes an amount of the composition which is capable of inducing an immune response in a subject upon administration of the composition to the subject.

Further, the disclosed antibody, in an embodiment may be used for diagnosing TNFRSF17, including cells expressing TNFRSF17.

The cells expressing TNFRSF17 typically include MM, CC and NHL cancer cells.

The diagnostic method comprises of contacting TNFRSF17 present in a sample with the antibody, specifically monoclonal antibody, of the present invention to form a complex, under conditions favorable for such complex formation, and detecting the complex formed by the antibody and TNFRSF17.

The disclosed antibody, in an embodiment may be provided in a diagnostic kit for detecting the presence of TNFRSF17, including cells expressing TNFRSF17.

The diagnostic kit for detecting the presence of TNFRSF17 in a sample comprises of antibodies that binds to a polypeptide sequence selected from the group consisting of SEQ ID NO: 1 and SEQ ID NO:2 in order to form a complex. The complex formed between the antibodies and the polypeptide sequence may further be detected by methods known in the art.

The “sample” may originate from a mammal, and may be a tissue sample (such as bone marrow tissue, colon tissue, etc.) or an extract of any tissue sample suspected of having cancer.

The detection of the complex as described herein can be performed by apparatus capable of detecting specific signals emitted by detectable labels generally known in the art such as radiation emission, color change, fluorescence, etc.

According to a non limiting aspect, the therapeutic monoclonal antibodies, TBL-CLN1, can be given as an adjuvant therapy either through intravenous infusion or in to the target tissue directly.

The invention is further defined by reference to the following example. It will be apparent to those skilled in the art that many modifications, both to materials and methods, may be practiced without departing from the scope of the invention.

### EXAMPLES

### Example 1

### Identification and Isolation of SEQ ID NO:1

TBL-CLN1 target was identified and validated over a period of 3 years by studying patient databases from numerous hospitals.

The identification and isolation of TNFRSF17 was carried out by:

a) obtaining around 20 cancer tissues from different hospitals,

b) isolating cancer cells using laser capture microscopy and,

c) conducting gene profile analysis.

### Example 2

### Production of Monoclonal Antibodies

Development of Myeloma cells: Mice were immunized with SEQ ID NO:2 with attached adjuvant. Mice serum was then screened using various techniques such as ELISA.

After sufficient titers were reached the mice were euthanized to remove the spleen as a source of cells for cell fusion. These cells were then treated with 8-azaguanine to ensure sensitivity to HAT.

Fusion procedure: Spleen cells harvested from mice were fused with myeloma cells. Fusion was done through co-centifusing in polyethylene glycol.

Cells were then plated in selection medium hypoxanthine-aminopterin-thymidine (HAT) selection medium-inhibitor of aminopterin which blocks nucleotide synthesis. Cells were distributed on feeder cells (murine bone-marrow) to promote growth of the hybridoma cells. Mice were then inoculated with the cells. The produced ascites was then collected from the mice and mAbs were screened using specific Ag binding activity.

The antibodies, isotype IgG1, having optimal binding/activity were identified. The antibodies were identified based on their ability to induced targeted ADCC and CDC mediated killing of cancer cells.

### Example 3

### Polynucleotide and Amino Acid Sequences of TBL-CLN1

The total RNA was extracted from hybridoma cells. The cDNAs encoding the variable region and constant region, including heavy and light chains, of the antibodies were amplified by PCR, cloned, and sequenced. The polypeptide and polynucleotide sequences of the heavy and light chains of TBL-CLN1 are as follows:

SEQ ID NO:3 and SEQ ID NO:5 are the amino acid sequences for the heavy chain and light chain of TBL-CLN1, respectively. SEQ ID NO:4 and SEQ ID NO:6 are the nucleotide sequence encoding the heavy chain and light chain of TBL-CLNI, respectively. SEQ ID NO:7 is the amino acid sequences of variable region of heavy chain of TBL-CLN1. SEQ ID NO:9 is the amino acid sequences of variable region of light chain of TBL-CLN1. SEQ ID NO:8 is the nucleotide sequence encoding the variable region of heavy chain of TBL-CLN1. SEQ ID NO:10 is the nucleotide sequence encoding the variable region of light chain of TBL-CLN1. The variable heavy chain CDR regions of TBL-CLN1 comprises of CDR1, CDR2 and CDR3 which has the amino acid sequence of SEQ ID NO:11, SEQ ID NO:12 and SEQ ID NO:13. The nucleotide sequence encoding CDR1, CDR2 and CDR3 are SEQ ID NO: 14, SEQ ID NO: 15 and SEQ ID NO: 16. The variable light chain CDR regions of TBL-CLN1 also comprises of CDR1, CDR2 and CDR3 which has the amino acid sequence of SEQ ID NO:17, SEQ ID NO:18 and SEQ ID NO:19. The nucleotide sequence encoding CDR1, CDR2 and CDR3 are SEQ ID NO: 20, SEQ ID NO: 21 and SEQ ID NO: 22. The constant heavy and light chain region of TBL-CLN1 has the amino acid sequence of SEQ ID NO:23 and SEQ ID NO:24, respectively. The nucleotide sequence encoding the constant heavy and light chain region of TBL-CLN1 are SEQ ID NO:25 and SEQ ID NO:26.

### Example 4

### Functional Characteristics of the Antibodies

ADCC Activity of TBL-CLN1: ADCC Assay

Procedure for Preparation of peripheral Blood Mononuclear Cells (PBMC): Blood from normal volunteer was collected in heparinized syringe. 10 mL of whole blood was transferred to a falcon tube. Blood and plasma was diluted to 30 mL in phosphate buffered saline (PBS) and mixed. 10 mL of room temp Ficoll Hypaque solution was then added to a 50 mL Falcon tube. Diluted blood was further overlaid on top of Ficoll Hypaque. Tubes were then centrifuged for 30 minutes, at room temperature (RT). PBMCs were collected from interphase of PBS/Ficoll and PBS was added. Cells were centrifuged at RT and supernatant was decanted. 5 mL RBC Lysis Solution was added to pellet and incubated for 5 min. at RT. Cells were diluted to 10 mL with PBS and centrifuged. Cells were then counted as per standard procedure. The cells were resuspended in assay medium containing fetal bovine serum (FBS), glutamine, HEPES, and a known antibiotic such as Penicillin and/or Streptomycin etc for ADCC assay.

The cancerous cell line was revived from the frozen stock stored in liquid nitrogen. The cancerous cell line was cultured with 10% serum containing a known medium. After reaching the required confluence the cells were collected and washed with a known medium. The cells were then centrifuged and the assay medium decanted.

Cancerous cell line was resuspended in assay medium with a specified cell density. Four specific known controls were employed in the assay a) PBMC control, b) Cancer cell control, c) Total cell lysis control and d) Background Control.

The samples were assayed in 96 well microtitre plate using serial dilution method. The plate was incubated for 2-6 hrs at 25° C.-40° C. temperature in an incubator to facilitate Antibody dependent cell lysis.

After incubation, the plate was centrifuged at RT and the supernatants were harvested. The harvested supernatants were then analyzed for LDH activity by using a commercial LDH detection kit.

The percent ADCC was calculated with the following equation:

Percentage CDC=((O.D of test−O.D of PBMC control-O.D of cancer cell control)/(O.D of total cell lysis−O.D of Cancer cell control))×100

The activity of the test antibody was examined by plotting the % ADCC activity against the log of antibody concentration using a four parameter curve fitting program.

Results of direct cytotoxicity and dose dependent assays against different cell lines are as follows:

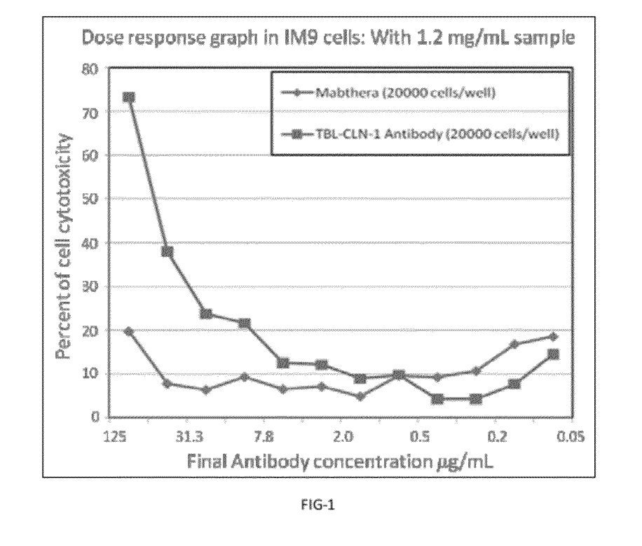

FIG. 1 is a graph depicting the results of direct cytotoxicity in IM9 cells with 1.2 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

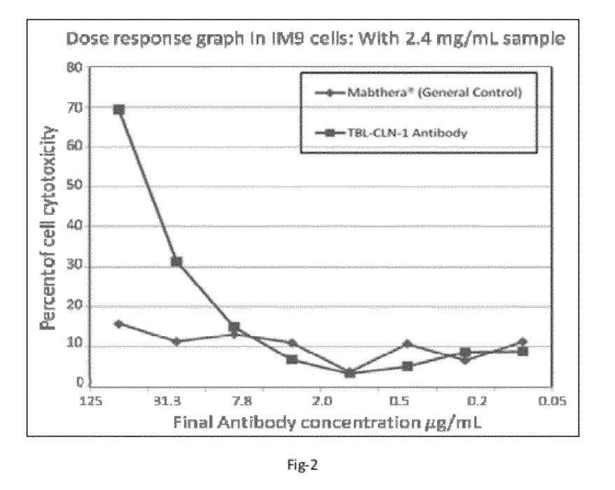

FIG. 2 is a graph depicting the results of direct cytotoxicity in IM9 cells with 2.4 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

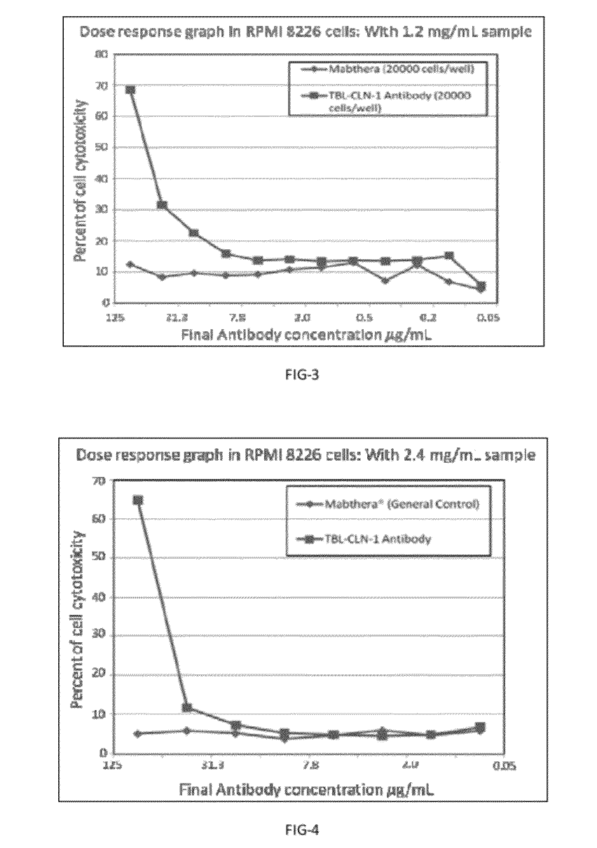

FIG. 3 is a graph depicting the results of direct cytotoxicity in RPMI-8226 cells with 1.2 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

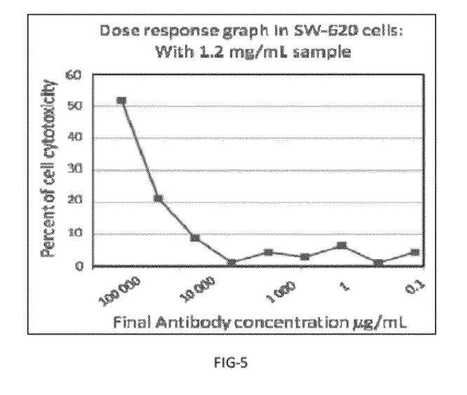

FIG. 4 is a graph depicting the results of direct cytotoxicity in RPMI-8226 cells with 2.4 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

FIG. 5 is a graph depicting the results of direct cytotoxicity in SW620 cell line with 1.2 mg/mL of TBL-CLN1 sample. The graph depicts the percentage cell cytotoxicity exhibited by TBL-CLN1 antibody at various TBL-CLN1 sample concentrations.

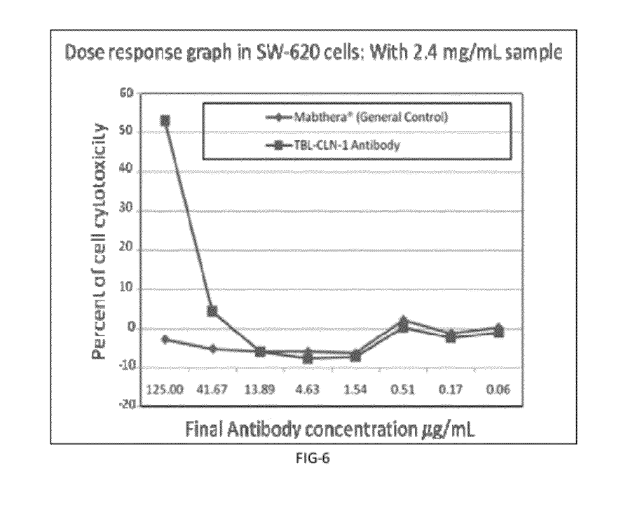

FIG. 6 is a graph depicting the results of direct cytotoxicity in SW620 cell line with 2.4 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

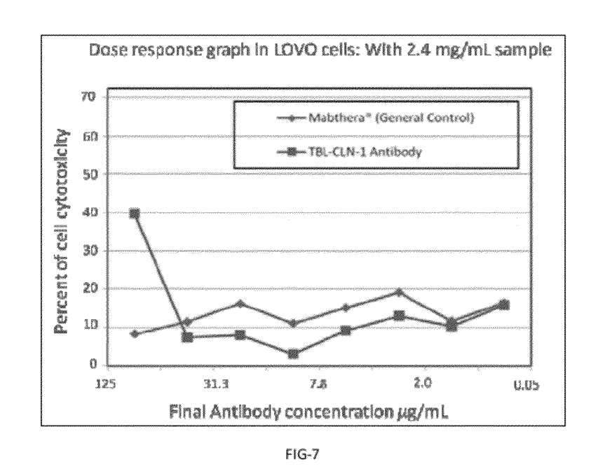

FIG. 7 is a graph depicting the results of direct cytotoxicity in LOVO cell line with 2.4 mg/mL of TBL-CLN1 sample. The control used was Mabthera®. The graph depicts the percentage cell cytotoxicity exhibited by TBL-CLN1 antibody at various TBL-CLN1 sample concentrations, which is plotted against the control.

CDC Activity of TBL-CLN1: CDC Assay

Procedure: The cancerous cell line was cultured in a tissue culture flask with serum containing growth medium. After reaching a targeted confluence, the cells were collected and counted. Ringer-Hepes buffer (RHB buffer) was prepared as per methods known in the art (the composition includes 20 mM HEPES, 2 mM glutamine, BSA and known antibiotics such as penicillin, streptomycin etc).

The cells were washed with RHB buffer and centrifuged. The buffer was then decanted. The cells were diluted to a density of 1 million cells/ml.

The samples were arranged in 96 wells of microtitre plate employing serial dilution method using four different controls. 50 μl of human serum complement was added to all the wells except the medium control, total lysis control and antibody control. 50 μl of 0.4% NP40 was further added to all the wells of column 11. 50 μl of cell suspension was then added to all the wells. After adding all the components, the plate was incubated in a CO₂ incubator at a range between 2-10% at a temperature between 30° C.-40° C.

After incubation, a known quantity of XTT was added to the wells and incubated for 2-10 hrs with intermittent shaking for few minutes. After incubation, readings were taken in an ELISA plate reader at 450 nm wavelength.

CDC was calculated with the following equation:

Percentage CDC=(O.D of test−O.D of Complement control)/(O.D of total cell lysis−O.D of complement control)×100

The activity of the test antibody was examined by plotting the % CDC activity against the log of antibody concentration using a four parameter curve fitting program.

The results of CDC assays demonstrating minimal CDC effect against different cell lines using a control are as follows:

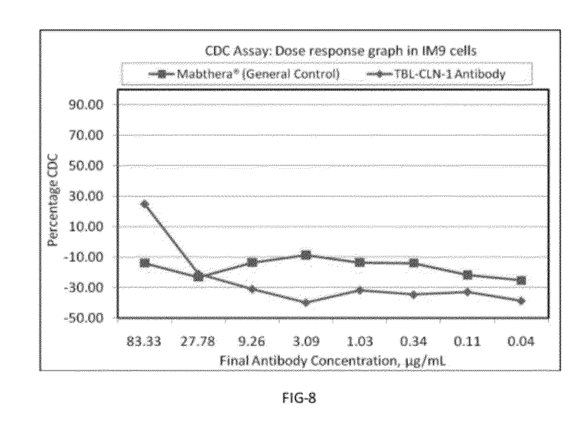

FIG. 8 is a graph depicting the results of complement dependent cytotoxicity in IM9 cell line. The control used was Mabthera®. The graph depicts the percentage CDC activity exhibited by TBL-CLN1 antibody at various TBL-CLN1 sample concentrations, which is plotted against control.

FIG. 9 is a graph depicting the results of complement dependent cytotoxicity in RPMI-8226 cell line. The control used was Mabthera®. The graph depicts the percentage CDC activity exhibited by TBL-CLN1 antibody at various TBL-CLN1 sample concentrations, which is plotted against control.

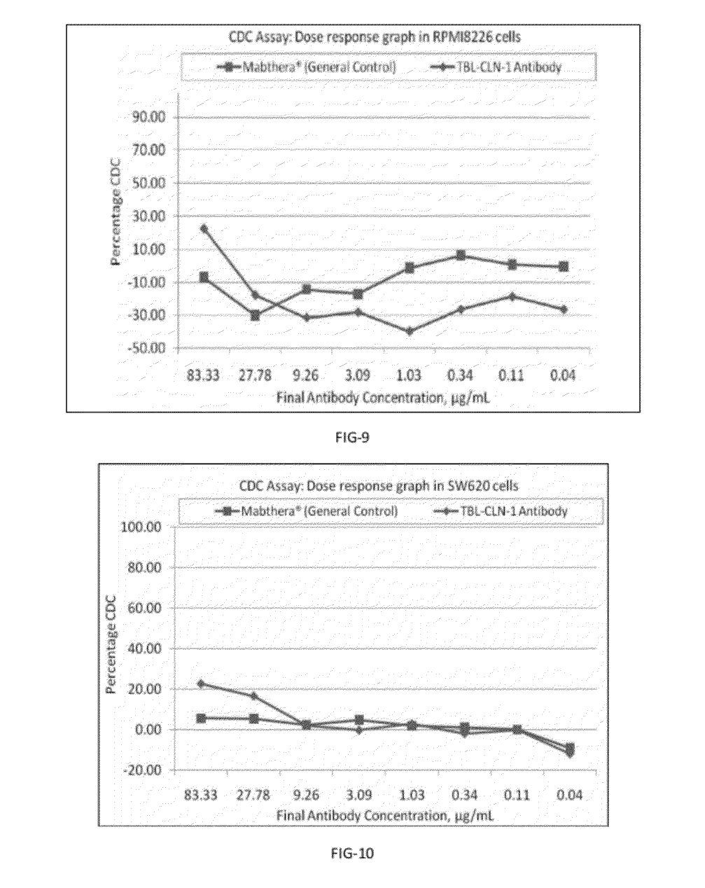

FIG. 10 is a graph depicting the results of complement dependent cytotoxicity in SW620 cell line. The control used was Mabthera®. The graph depicts the percentage CDC activity exhibited by TBL-CLN1 antibody at various TBL-CLN1 sample concentrations, which is plotted against control.

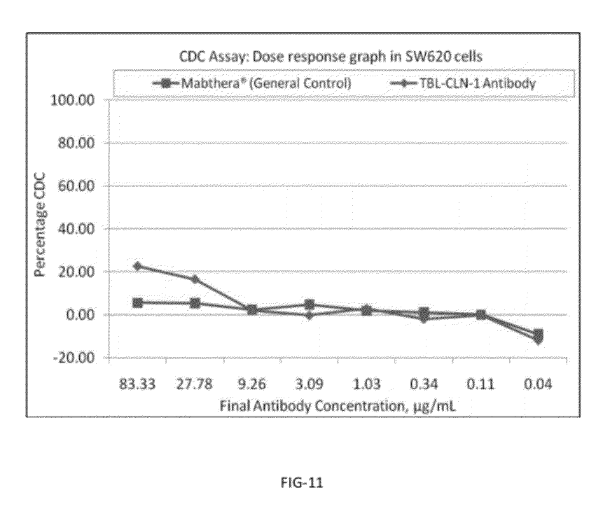

FIG. 11 is a graph depicting the results of complement dependent cytotoxicity in LOVO cell line. The control used was Mabthera®. The graph depicts the percentage CDC activity exhibited by TBL-CLN1 antibody at various TBL-CLN1 sample concentrations, which is plotted against control.

The cytotoxicity further demonstrated a reasonable titre of mouse antibody of LD50<50 ug/ml.

When injected to SCID mice with human myeloma xenograft, the antibody specifically bound to the cell surface antigen of multiple myeloma cancer cells initiating ADCC and apoptosis to kill the cancer cells.

Significant cytotoxicity was observed towards the target cancer cells. The results of the assays demonstrated that the antibody (TBL-CLN1) has cytotoxicity towards specific cancer cell lines. Binding assays confirmed TBL-CLN1's cytotoxicity effect against Non-Hodgkins Lymphoma, Multiple Myeloma, and Colon cancers.

Humanization was performed in order to generate chimeric antibody, humanized antibody and de-immunized antibody. The activity of humanized antibody was tested at each step to make sure original cytotoxicity was retained.

### Example 5

### Humanized Antibodies

Humanized antibody expressed in microbial/murine cell lines. The humanized monoclonal antibody was cloned and expressed in the selected and genetically modified microbial and/or murine cells. The methods for production and purification of the expressed/secreted protein are known in the art. The production and purification of the expressed/secreted protein was carried out in a bioreactor providing optimized conditions such as pH, DO, CO₂, air etc.

The expressed protein was then purified through series of steps such as centrifugation, chromatography systems etc to arrive at the purified protein, humanized monoclonal antibodies.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A polypeptide sequence of SEQ ID NO: 2.

2. The polypeptide sequence of claim 1, wherein said polypeptide sequence is capable of being administered in an immunologically sufficient amount for eliciting an immune response in a mammalian subject.

3. An antibody that specifically binds to a polypeptide sequence selected from the group consisting of SEQ ID NO: 1 and SEQ ID NO: 2.

4. The antibody as claimed in claim 3, wherein said antibody is humanized to generate chimeric antibody, humanized antibody and de-immunized antibody.

5. The antibody as claimed in claim 3, comprising:
a heavy chain polypeptide sequence of SEQ ID NO:3; and
a light chain polypeptide sequence of SEQ ID NO:5.

6. The antibody as claimed in claim 3, comprising:
a variable heavy chain polypeptide sequence of SEQ ID NO:7; and
a variable light chain polypeptide sequence of SEQ ID NO:9.

7. The antibody as claimed in claim 3, comprising at least one CDR sequence selected from the group consisting of variable heavy chain CDR1 of SEQ ID NO:11, variable heavy chain CDR2 of SEQ ID NO:12, variable heavy chain CDR3 of SEQ ID NO:13, variable light chain CDR1 of SEQ ID NO:17, variable light chain CDR2 of SEQ ID NO:18 and variable light chain CDR3 of SEQ ID NO:19.

8. The antibody as claimed in claim 3, wherein said antibody is a monoclonal antibody.

9. The antibody as claimed in claim 3, wherein said antibody is selected from the group consisting of IgG1, IgG2, IgG3, IgG4, IgM, IgA1, IgA2, secretory IgA, IgD, and IgE antibody.

10. The antibody as claimed in claim 3, wherein said antibody has at least one of the characteristics selected from the group consisting of:
capable of inducing complement dependent cytotoxicity (CDC) of cancer cells expressing TNFRSF17;
capable of inducing apoptosis of cancer cells expressing TNFRSF17; and
capable of inducing antibody dependent cellular cytotoxicity (ADCC) of cancer cells expressing TNFRSF17;

11. The antibody as claimed in claim 3, wherein said antibody is expressed in a vector, wherein the vector comprises of a polynucleotide sequence of SEQ ID NO:8 and SEQ ID NO:10.

12. The antibody as claimed in claim 11, wherein said vector is transfected into a cell line.

13. The antibody as claimed in claim 12, wherein said cell line is a microbial cell line.

14. The antibody as claimed in claim 3, wherein said antibody is present in a pharmaceutical composition.

15. The antibody as claimed in claim 14, wherein said pharmaceutical composition further comprises of a pharmaceutically acceptable carrier, wherein the pharmaceutically acceptable carrier is selected from the group consisting of water, salt solutions, gelatins, oils, alcohols and mixtures thereof.

16. The antibody as claimed in claim 3, wherein said antibody is capable of being administered in a therapeutically effective amount for treating of cancer.

17. The antibody as claimed in claim 16, for the treatment of cancer, wherein said cancer expresses a protein with a region comprising a polypeptide sequence selected from the group consisting of SEQ ID NO: 1 and SEQ ID NO:2.

18. The antibody as claimed in claim 16, for the treatment of cancer, wherein said cancer is selected from the group consisting of Multiple Myeloma, Colon Cancer and Non-Hodgkin's Lymphoma.

19. A method for diagnosing the presence of TNFRSF17 in a sample, said method comprising:
contacting said sample with antibody under conditions that allow for formation of a complex between said antibody and TNFRSF17, wherein said antibody specifically binds to a polypeptide sequence selected from the group consisting of SEQ ID NO: 1 and SEQ ID NO:2; and
detecting the formation of said complex.
